(19) United States
(12) Patent Application Publication
(10) Pub. No.: US 2015/0360037 A1
(43) Pub. Date: Dec. 17, 2015

(54) LEADS, SYSTEMS, AND METHODS USING EXTERNAL PRIMARY AND INTERNAL SECONDARY POWER SOURCES

(71) Applicant: Boston Scientific Neuromodulation Corporation, Valencia, CA (US)

(72) Inventors: Benjamin Phillip Hahn, Stevenson Ranch, CA (US); Que T. Doan, West Hills, CA (US)

(21) Appl. No.: 14/734,777

(22) Filed: Jun. 9, 2015

Related U.S. Application Data
(60) Provisional application No. 62/012,141, filed on Jun. 13, 2014.

(51) Int. Cl. A61N 1/378; A61N 1/372

(52) U.S. Cl. CPC A61N 1/3787; A61N 1/37229

(57) ABSTRACT

An electrical stimulation system includes an implantable control module for implantation in a body of a patient and having an antenna, a secondary power source, and a processor coupled to the antenna and the secondary power source. The control module provides electrical stimulation current to an electrical stimulation lead for stimulation of patient tissue. The system also includes a primary power source to be worn or carried by the patient external to the body of the patient and to deliver power to the control module through the antenna. The control module preferentially utilizes power directly from the primary power source for the electrical stimulation current when the primary power source is available. The system can also include an electrical stimulation lead, a lead extension, or an external programming unit.

### CROSS-REFERENCE TO RELATED APPLICATIONS

This application claims the benefit under 35 U.S.C. §119(e) of U.S. Provisional Patent Application Ser. No. 62/012,141, filed Jun. 13, 2014, which is incorporated herein by reference.

### FIELD

The present invention is directed to the area of implantable electrical stimulation systems and methods of making and using the systems. The present invention is also directed implantable electrical stimulation systems that use an external primary power source and an internal secondary power source, as well as methods of making and using the electrical stimulation systems.

### BACKGROUND

Implantable electrical stimulation systems have proven therapeutic in a variety of diseases and disorders. For example, spinal cord stimulation systems have been used as a therapeutic modality for the treatment of chronic pain syndromes. Peripheral nerve stimulation has been used to treat chronic pain syndrome and incontinence, with a number of other applications under investigation. Functional electrical stimulation systems have been applied to restore some functionality to paralyzed extremities in spinal cord injury patients.

Stimulators have been developed to provide therapy for a variety of treatments. A stimulator can include an implantable pulse generator (IPG), one or more leads, and an array of stimulator electrodes on each lead. The stimulator electrodes are in contact with or near the nerves, muscles, or other tissue to be stimulated. The pulse generator generates electrical pulses that are delivered by the electrodes to body tissue.

### BRIEF SUMMARY

One embodiment is an electrical stimulation system that includes an implantable control module for implantation in a body of a patient and having an antenna, a secondary power source, and a processor coupled to the antenna and the secondary power source. The control module provides electrical stimulation current to an electrical stimulation lead for stimulation of patient tissue. The system also includes a primary power source to be worn or carried by the patient external to the body of the patient and to deliver power to the control module through the antenna. The control module preferentially utilizes power directly from the primary power source for the electrical stimulation current when the primary power source is available.

In at least some embodiments, secondary power source has a storage capacity that is no more than 25% of a storage capacity of the primary power source. In at least some embodiments, the secondary power source has a storage capacity that is no more than 10% of a storage capacity of the primary power source.

In at least some embodiments, the electrical stimulation system also includes an external antenna extending from the primary power source and having a distal end portion; an external fixation element coupled to the distal end portion of the external antenna; and an internal fixation element coupled to the implantable control module or the antenna of the control module where the external and internal fixation elements are configured and arranged to hold the distal end portion of the external antenna to a skin of a patient at a preselected placement position when the internal fixation element is implanted in the patient near the preselected placement position. In at least some embodiments, the internal and external fixation elements each comprise a magnet. In at least some embodiments, the control module also includes a housing with the antenna extending away from the housing, the antenna have a distal end portion, where the internal fixation element is disposed on a distal end portion of the antenna.

Another embodiment is an electrical stimulation system that includes an implantable control module for implantation in a body of a patient and having an antenna, a secondary power source, and a processor coupled to the antenna and the secondary power source. The control module provides electrical stimulation current to an electrical stimulation lead for stimulation of patient tissue. The system also includes a primary power source to be worn or carried by the patient external to the body of the patient and to deliver power to the control module through the antenna. The control module utilizes power directly from the primary power source for the electrical stimulation current and the secondary power source has a storage capacity that is no more than 25%, or no more than 10%, of a storage capacity of the primary power source.

A further embodiment is an electrical stimulation system that includes an implantable control module for implantation in a body of a patient and having an antenna, a secondary power source, and a processor coupled to the antenna and the secondary power source. The control module provides electrical stimulation current to an electrical stimulation lead for stimulation of patient tissue. The system also includes a primary power source to be worn or carried by the patient external to the body of the patient and to deliver power to the control module through the antenna. The control module utilizes power directly from the primary power source for the electrical stimulation current. The system further includes an external antenna extending from the primary power source and comprising a distal end portion; an external fixation element coupled to the distal end portion of the external antenna; and an internal fixation element coupled to the implantable control module or the antenna of the control module. The external and internal fixation elements hold the distal end portion of the external antenna to a skin of a patient at a preselected placement position when the internal fixation element is implanted in the patient near the preselected placement position. The external antenna, external fixation element, and internal fixation element can also be used with any of the other systems described above.

In at least some embodiments, the internal and external fixation elements each include a magnet. In at least some embodiments, the control module further includes a housing with the antenna extending away from the housing, the antenna have a distal end portion, wherein the internal fixation element is disposed on a distal end portion of the antenna.

Any of the systems described above can also include an electrical stimulation lead coupleable, or coupled, to the control module and has at least one lead body having a distal end portion and a proximal end portion, electrodes disposed along the distal end portion of the at least one lead body, terminals disposed along the proximal end portion of the at least one lead body, and conductors electrically coupling the terminals to the electrodes.

Any of the systems described above can also include a lead extension coupleable between the electrical stimulation lead and the control module.

Any of the systems described above can also include an external programming unit configured and arranged for programming or modifying a set of stimulation parameters in the processor of the control module.

Any of the systems described above can also include an electrical stimulation lead comprising at least one lead body having a distal end portion, electrodes disposed along the distal end portion of the at least one lead body, and conductors electrically coupling the electrodes to the electronic subassembly of the control module.

In at least some embodiments of any of the systems described above, the secondary power source is configured and arranged to provide no more than four hours, or no more than two hours, of continuous electrical stimulation current when fully charged. In at least some embodiments of any of the systems described above, the control module defines a port for receiving a proximal end of the electrical stimulation lead. In at least some embodiments of any of the systems described above, the primary power source includes a processor configured and arranged to provide electrical stimulation current based on a set of stimulation parameters. In at least some embodiments of any of the systems described above, the control module is configured and arranged to solely utilize power directly from the primary power source for the electrical stimulation current when the primary power source is available.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic block diagram of one embodiment of an electrical stimulation system, according to the invention;

FIG. 2 is a schematic block diagram of another embodiment of an electrical stimulation system, according to the invention;

FIG. 3A is a schematic diagram of one embodiment of an arrangement of an external primary power source and a control module with wireless transmission, according to the invention;

FIG. 3B is a schematic diagram of a second embodiment of an arrangement of an external primary power source and a control module with a wired connection, according to the invention;

FIG. 3C is a schematic diagram of a third embodiment of an arrangement of an external primary power source and a control module with an antenna extending from the control module, according to the invention;

FIG. 3D is a schematic diagram of a fourth embodiment of an arrangement of an external primary power source and a control module with antennas extending from the control module and the primary power source, according to the invention;

FIG. 3E is a schematic diagram of a fifth embodiment of an arrangement of an external primary power source and a control module with an antenna extending from the primary power source, according to the invention;

FIG. 4 is a schematic side view of one embodiment of an electrical stimulation system that includes a paddle lead electrically coupled to an implantable control module, according to the invention:

FIG. 5 is a schematic side view of one embodiment of an electrical stimulation system that includes a percutaneous lead electrically coupled to an implantable control module, according to the invention;

FIG. 6A is a schematic side view of one embodiment of the implantable control module of FIG. 4 configured and arranged to electrically couple to an elongated device, according to the invention; and

FIG. 6B is a schematic side view of one embodiment of a lead extension configured and arranged to electrically couple the elongated device to the implantable control module of FIG. 4, according to the invention.

### DETAILED DESCRIPTION

The present invention is directed to the area of implantable electrical stimulation systems and methods of making and using the systems. The present invention is also directed implantable electrical stimulation systems that use an external primary power source and an internal secondary power source, as well as methods of making and using the electrical stimulation systems.

Suitable implantable electrical stimulation systems include, but are not limited to, a least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741.892; 7,949,395; 7,244.150; 7,672,734; 7,761,165; 7,974,706; 8,175,710; 8,224,450; and 8,364,278; and U.S. Patent Application Publication No. 2007/0150036, all of which are incorporated by reference.

In many conventional electrical stimulation systems, the lead is coupled to an implantable control module (e.g., an implantable pulse generator) that includes a primary power source that can be recharged and is suitable for long term (e.g., more than six months, one year, five years, or longer) use. The primary power source can be a battery, capacitor, or the like. Such power sources, however, are relatively large when compared to other components of the implantable control module and necessitate implanting the control module away from the stimulation site in a portion of the body, such as the buttocks or large body cavity, where there is sufficient space for the control module. The power source is often the largest element in the control module.

In contrast to conventional electrical stimulation systems, an electrical stimulation system can include a primary power source that is wearable by, and external to, the user and which transmits or otherwise delivers energy to the implanted control module or lead to provide stimulation current to the tissue. For example, the primary power source can be configured to be worn on the belt of the user; around the wrist of the user; clipped to pants, shirt, or other item of clothing of the patient; or otherwise carried; or any other suitable method of wearing or carrying the primary power source. The implanted control module or lead includes a secondary power source that can temporarily generate stimulation current when the primary power source is removed, discharged, or otherwise unavailable.

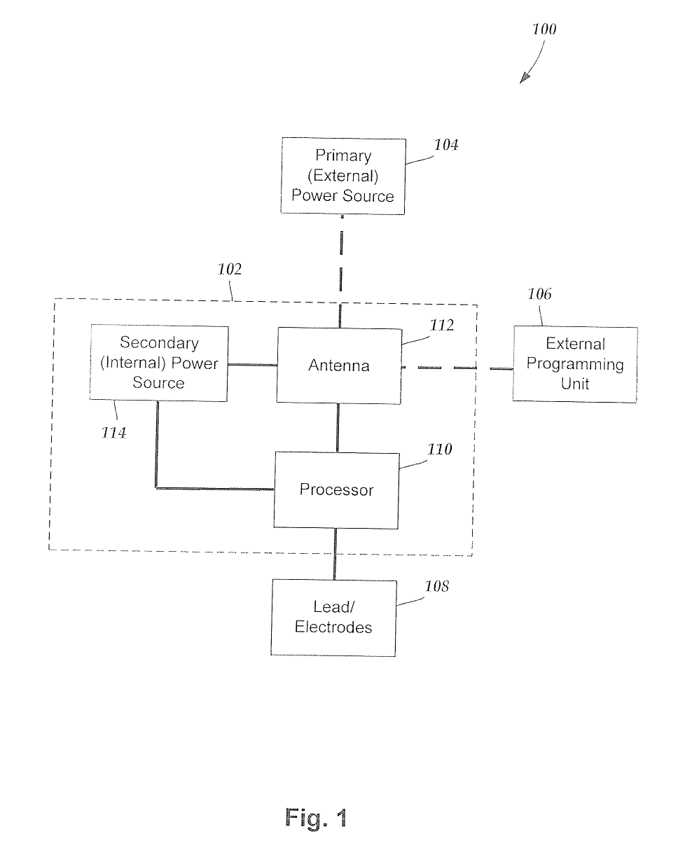

FIG. 1 illustrates schematically one embodiment of an electrical stimulation system 100 that includes an implantable control module (e.g., a stimulator or pulse generator) 102, one or more leads 108 with electrodes, an external primary power source 104, and an external programming unit 106. In at least some embodiments, the external power source 104 and external programming unit 106 can be combined in a single device. In other embodiments, the external power source 104 and external programming unit 106 can be separate devices.

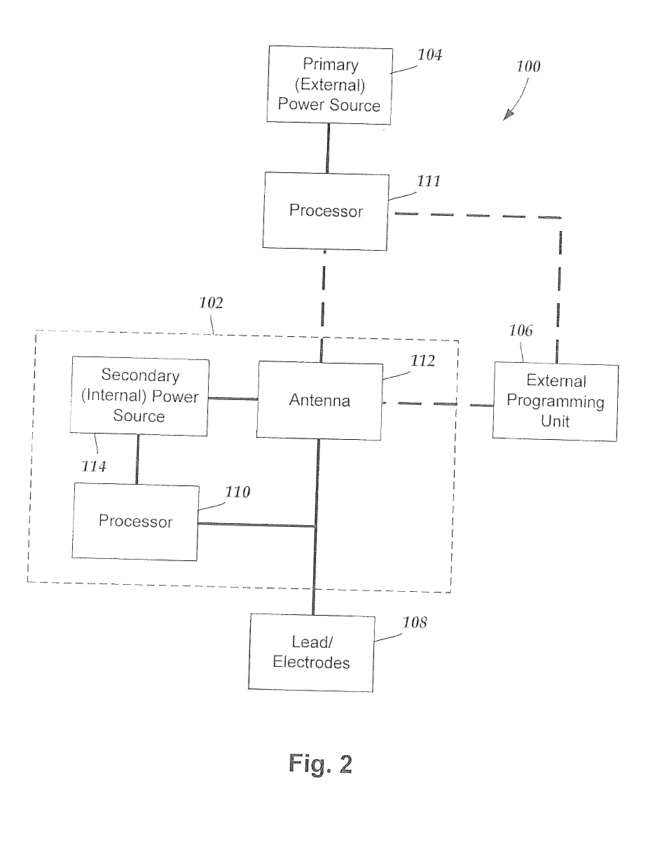

The lead 108 is coupled, or coupleable, to the implantable control module 102. The implantable control module 102 includes a secondary power source 114, a processor 110, and an antenna 112. It will be understood that the electrical stimulation system can include more, fewer, or different components and can have a variety of different configurations including those configurations disclosed in the stimulator references cited herein. For example, FIG. 2 illustrates an alternative arrangement with an external processor 111, described below, coupled to the primary power source 104.

The primary power source 104 and the external programming unit 106 are not implanted within the patient. The primary power source 104 can provide energy to the processor 110 and lead 108 by wireless transmission through the antenna 112. The primary power source 104 can also be used to recharge the second power source 114 through the antenna 112. In at least some embodiments, the primary power source 104 directly powers the processor 110 through the antenna 112. The external programming unit 106 can be used to set or modify stimulation parameters stored by the processor 110 and used to determine the characteristics of the stimulation current provided to the tissue through the lead 108.

Any primary power source 104 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bio-energy power sources, fuel cells, bioelectric cells, osmotic pressure pumps, and the like including the power sources described in U.S. Pat. No. 7,437,193, incorporated herein by reference.

The primary power source 104 can be rechargeable. In some embodiments, the primary power source 104 can be recharged wirelessly. In some embodiments, the primary power source 104 can be recharged by attachment to a wall socket or other recharging source. The patient may have two or more primary power sources so that the can exchange one primary power source for the other when, for example, the first is being recharged.

Any suitable, small power source can be used for the secondary power source 114 including, but not limited to, a rechargeable battery or super capacitor. The secondary power source 114 typically has a relatively small amount of stored energy compares to the primary power source 104. In some embodiments, the secondary power source 114 has a storage capacity of no more than 10%, 15%, or 25% of the storage capacity of the primary power source 104. In some embodiments, the secondary power source 114 is configured and arranged to only supply continuous (pulsed or non-pulsed) stimulation current to the lead for no more than one hour, two hours, four hours, six hours, eight hours, twelve hours, or twenty-four hours. In some embodiments, the secondary power source 114 is configured and arranged to only supply the programmed stimulation current to the lead for no more than one hour, two hours, four hours, six hours, eight hours, twelve hours, or twenty-four hours. The secondary power source 114 can be useful for temporary operation of the electrical stimulation system 100 such as, for example, when the patient bathes, showers, or engages in athletic or other activities in which wearing the primary power source 104 is uncomfortable or undesirable.

In at least some embodiments, the system 100 is configured and arranged so that the control module 102 draws power solely from the primary power source 104 unless the primary power source 104 is unavailable, in which case, the control module draws power from the secondary power source 114. In other embodiments, the system 100 is configured and arranged so that the control module 102 draws power from the secondary power source 114 unless the secondary power source 114 is unavailable or discharged or discharged below a preselected threshold amount, in which case, the control module draws power from the primary power source 104. In yet other embodiments, the system 100 is configured and arranged so that the control module 102 draws power from both the primary power source 104 and the secondary power source 114. In some embodiments, the system 100 is configured and arranged so that the user or the programmer (or both) can switch between two or more of the power drawing configurations described in this paragraph.

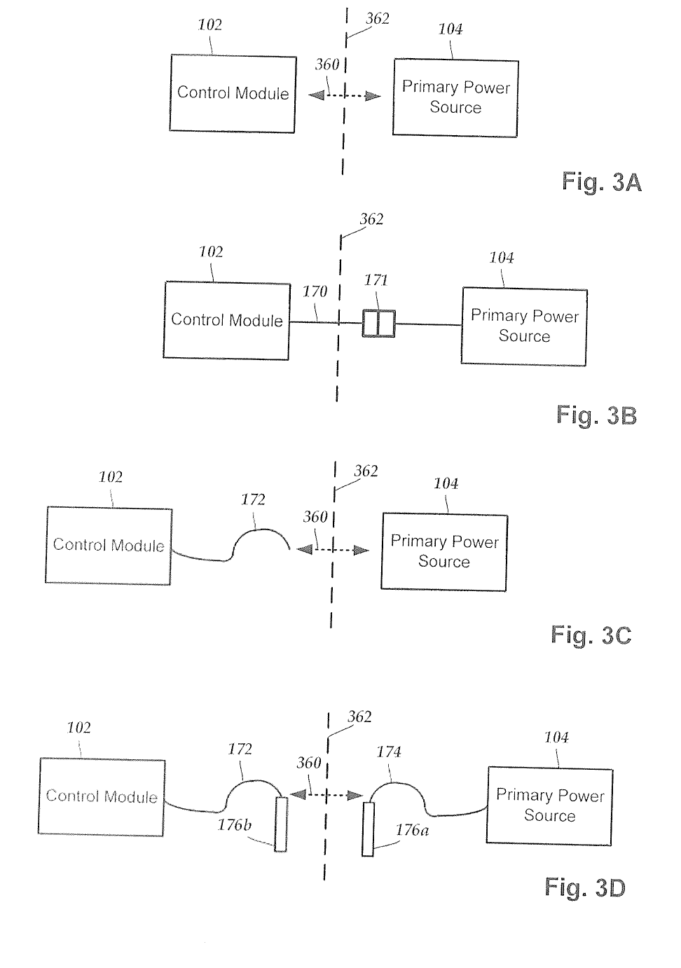

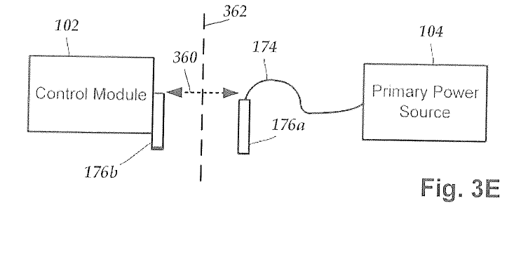

Power is provided to the control module 102 by the primary power source 104 through wireless transmission (e.g., RF transmission or inductive coupling) via the antenna 112. FIG. 3A illustrates a wireless connection 360 across the skin boundary 362 between the control module 102 and the primary power source 104. The antenna 112 (see. FIG. 2), or any other antenna described herein, can have any suitable configuration including, but not limited to, a coil, looped, or loopless configuration, or the like. In some embodiments, power is transmitted at a frequency of at least 50 kHz, 80 kHz, 100 kHz, or higher. In some embodiments, the power is transmitted at a frequency of at least 1 MHz, 5 MHz, or higher. In at least some embodiments, a higher transmission frequency will facilitate transmission over a longer distance. A transmission frequency may be selected based on government regulations, interference from other sources, or any combination of these and other factors.

The primary power source 104 and external programming unit 106 will typically also each include an antenna to transmit to, or receive transmission from, the control module 102. The antennas of the control module 102, primary power source 104, and external programming unit 106 may be designed for a particular transmission frequency or frequencies and there may be separate antennas, designed for different transmission frequencies, in the control module to communicate individually with the primary power source and the external programming unit.

As an alternative, the primary power source 104 can be coupled to the control module 102 by a cable 170 that extends into the patient, as illustrated in FIG. 3B. The cable may include an external connector 171 that allows the primary power source 104 to be uncoupled from the control module.

FIG. 3C illustrates another alternative in which an antenna 172 extends from a housing of the control module 102 and has a distal end positioned at a preselected placement position near the skin 362 for communication with the primary power source 104. It will be understood that the antenna 172, in this and any other embodiment described herein, may also incorporate any of the other elements of the control module 102 such as the processor 110 (or a portion of the processor) or the secondary power source 114.

FIG. 3D illustrates a further embodiment in which an internal antenna 172 extends from a housing of the control module 102 and has a distal end positioned at a preselected placement position near the skin 362 for communication with an external antenna 174 extending from the primary power source 104. In this embodiment, optional fixation elements 176a, 176b, such as magnets, may be provided so that the external antenna 174 can be attached to the skin 362 near the internal antenna 172.

FIG. 31E illustrates yet another embodiment in which an antenna 174 extends from the primary power source 104 for communication with the control module 102. In this embodiment, optional fixation elements 176a, 176b, such as magnets, may be provided so that the antenna 174 can be attached to the skin 362 near the control module 102.

Returning to FIG. 1, in at least some embodiments, the processor 110 is configured to control one or more of the timing, frequency, strength, duration, and waveform of the pulses. In addition, the processor 110 can select which electrodes of the lead 108 can be used to provide stimulation, if desired. In some embodiments, the processor 110 selects which electrode(s) are cathodes and which electrode(s) are anodes. In some embodiments, the processor 110 is used to identify which electrodes provide the most useful stimulation of the desired tissue.

Any processor 110 can be used and can be as simple as an electronic device that, for example, produces pulses at a regular interval or the processor can be capable of receiving and interpreting instructions from the external programming unit 106 that, for example, allows modification of pulse characteristics. In the illustrated embodiment, the processor 110 is coupled to the antenna 112 to receive signals from the external programming unit 106.

Turning to FIG. 2, in alternative embodiments, the primary power source 104 includes a processor 111 that is similar to, or the same as, the processor 110 described above. The processor 111 can be used to generate the electrical stimulation current with desired stimulation parameters that are then delivered directly (or via the control module 102) to the lead 108. An optional second processor 110 can be included in the control module 102 to generate electrical stimulation current with desired stimulation parameters using the second power source 114. In some embodiments, both processors 110, 111 can be used to generate electrical stimulation current with desired stimulation parameters from the primary power source 104. The processor 111 and optional processor 110 can be programmed (for example, stimulation parameters set or modified) using the external programming unit 106. Alternatively or additionally, the processor 111 may be coupled to an input device that allows the stimulation parameters to be set or modified directly by a user without the external programming unit.

Returning to FIG. 1, the external programming unit 106 can be any unit that can provide information to the processor 110 or processor 111 via an antenna. One example of a suitable programming unit is a computer operated by the user or clinician to send signals to the control module 102. The signals sent to the processor 110 (or processor ill) via the antenna 112 can be used to modify or otherwise direct the operation of the electrical stimulation system. For example, the signals may be used to modify the pulses of the electrical stimulation system such as modifying one or more of pulse duration, pulse frequency, pulse waveform, and pulse strength. The signals may also direct the electrical stimulation system 100 to cease operation, to start operation, to start charging the internal power source 114, or to stop charging, the internal power source. In some embodiments, the primary power source 104 and the external programming unit 106 can form a single device.

Optionally, the electrical stimulation system 100 may include a transmitter (not shown) coupled to the processor 110 and the antenna 112 for transmitting signals back to the external programming unit or another unit capable of receiving the signals. For example, the electrical stimulation system 100 may transmit signals indicating whether the electrical stimulation system 100 is operating properly or not or indicating when the battery needs to be charged or the level of charge remaining in the battery. The control unit 102 may also be capable of transmitting information about the pulse characteristics so that a user or clinician can determine or verify the characteristics.

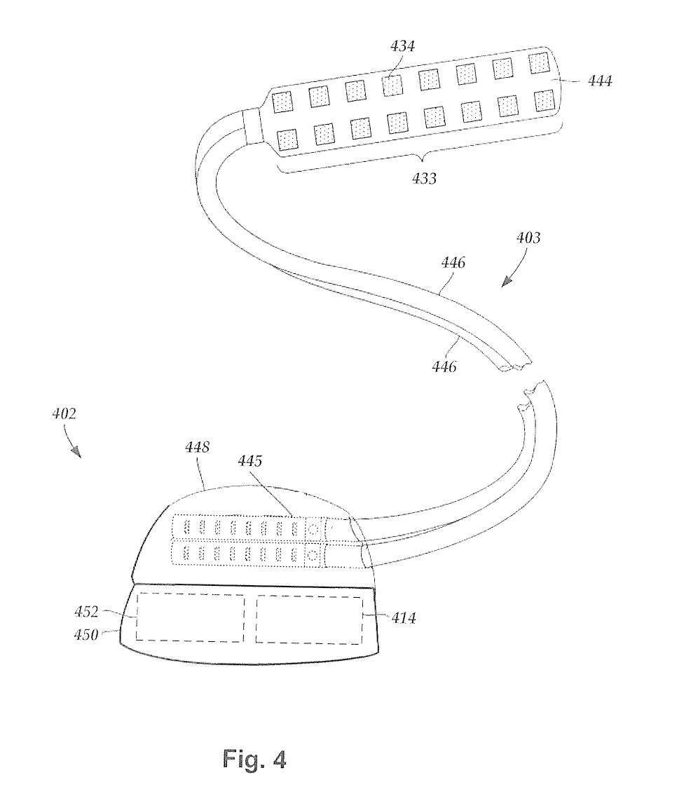

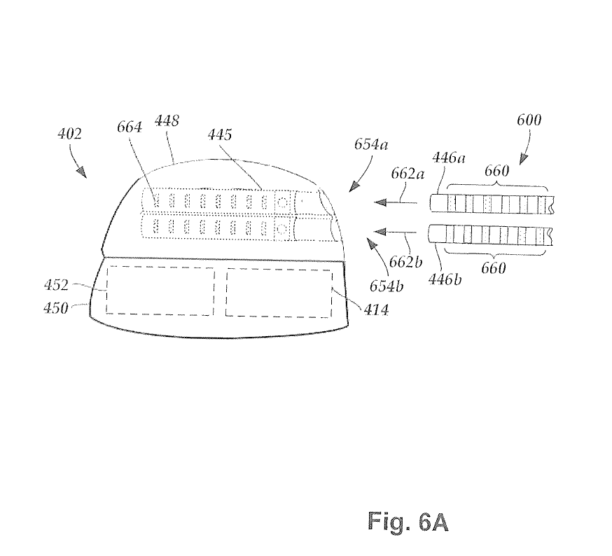

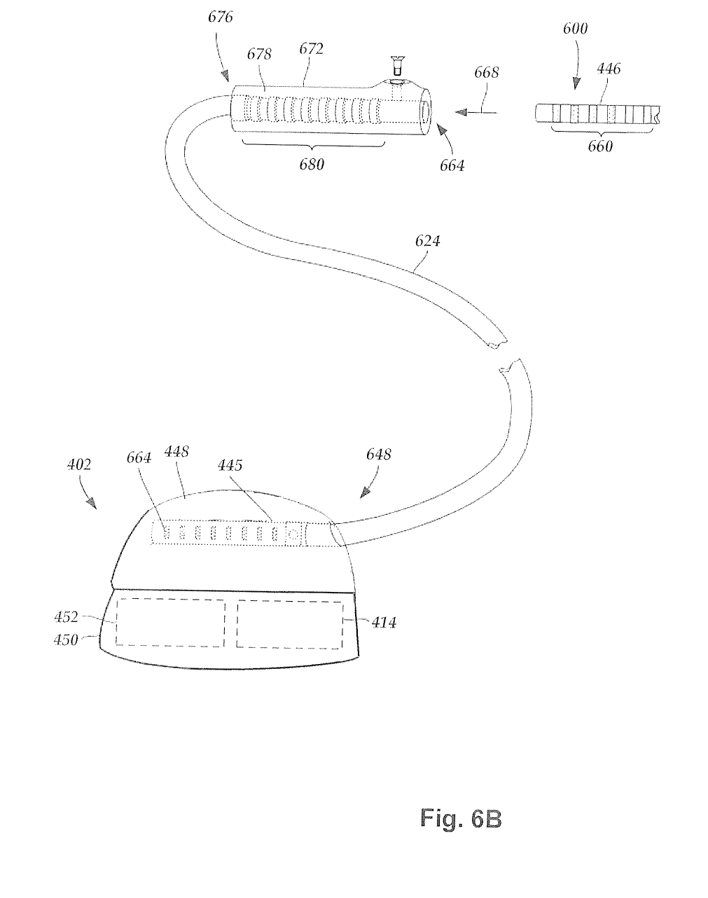

FIG. 4 illustrates one embodiment of a control module 402 and lead 403. The lead 403 includes a paddle body 444 and one or more lead bodies 446. In FIG. 4, the lead 403 is shown having two lead bodies 446. It will be understood that the lead 403 can include any suitable number of lead bodies including, for example, one, two, three, four, five, six, seven, eight or more lead bodies 446. An array of electrodes 433, such as electrode 434, is disposed on the paddle body 444, and one or more terminals (e.g., 660 in FIGS. 6A and 6B) are disposed along each of the one or more lead bodies 446. In at least some embodiments, the lead has more electrodes than terminals.

It will be understood that the electrical stimulation system can include more, fewer, or different components and can have a variety of different configurations including those configurations disclosed in the electrical stimulation system references cited herein. For example, instead of a paddle body, the electrodes can be disposed in an array at or near the distal end of a lead body forming a percutaneous lead.

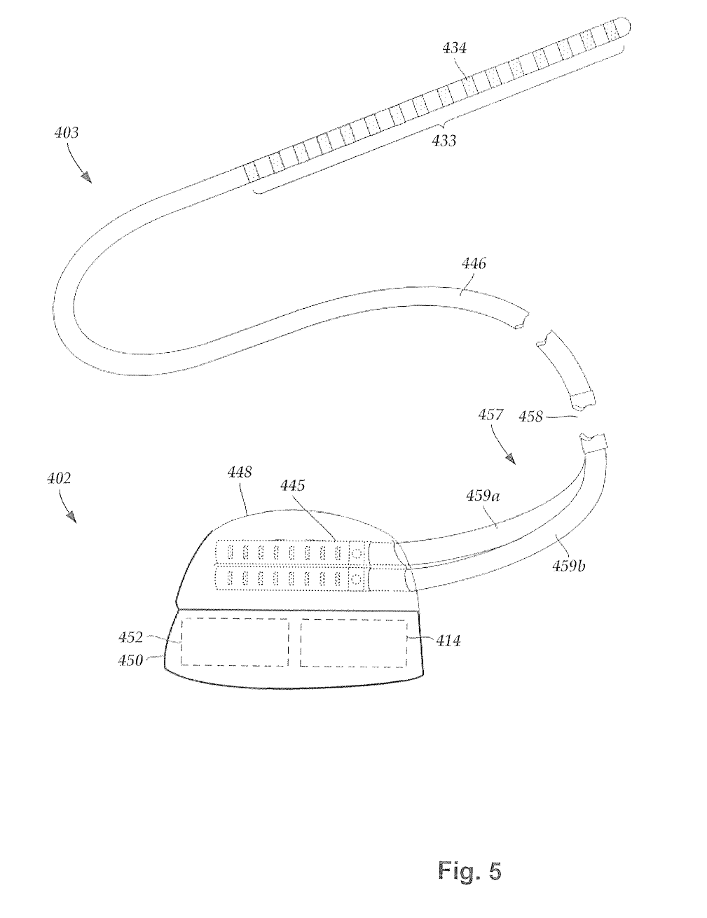

FIG. 5 illustrates schematically another embodiment in which the lead 403 is a percutaneous lead. In FIG. 5, the electrodes 434 are shown disposed along the one or more lead bodies 446. In at least some embodiments, the lead 403 is isodiametric along a longitudinal length of the lead body 446.

The lead 403 can be coupled to the implantable control module 402 in any suitable manner. In FIG. 4, the lead 403 is shown coupling directly to the implantable control module 402. In at least some other embodiments, the lead 403 couples to the implantable control module 402 via one or more intermediate devices (600 in FIGS. 6A and 6B). For example, in at least some embodiments one or more lead extensions 624 (see e.g., FIG. 6B) can be disposed between the lead 403 and the implantable control module 402 to extend the distance between the lead 403 and the implantable control module 402. Other intermediate devices may be used in addition to, or in lieu of, one or more lead extensions including, for example, a splitter, an adaptor, or the like or combinations thereof. It will be understood that, in the case where the electrical stimulation system includes multiple elongated devices disposed between the lead 403 and the implantable control module 402, the intermediate devices may be configured into any suitable arrangement.

In FIG. 5, the electrical stimulation system 400 is shown having a splitter 457 configured and arranged for facilitating coupling of the lead 403 to the implantable control module 402. The splitter 457 includes a splitter connector 458 configured to couple to a proximal end of the lead 403, and one or more splitter tails 459a and 459b configured and arranged to couple to the implantable control module 402 (or another splitter, a lead extension, an adaptor, or the like).

The implantable control module 402 includes a connector housing 448 and a sealed electronics housing 450. An electronic subassembly 452 (which includes the processor 110 (see. FIG. 1) and the secondary power source 414 are disposed in the electronics housing 450. A connector 445 is disposed in the connector housing 448. The connector 445 is configured and arranged to make an electrical connection between the lead 403 and the electronic subassembly 452 of the implantable control module 402.

The electrical stimulation system or components of the electrical stimulation system, including the paddle body 444, the one or more of the lead bodies 446, and the implantable control module 402, are typically implanted into the body of a patient. The electrical stimulation system can be used for a variety of applications including, but not limited to deep brain stimulation, neural stimulation, spinal cord stimulation, muscle stimulation, and the like.

The electrodes 434 can be formed using any conductive, biocompatible material. Examples of suitable materials include metals, alloys, conductive polymers, conductive carbon, and the like, as well as combinations thereof. In at least some embodiments, one or more of the electrodes 434 are formed from one or more of: platinum, platinum iridium, palladium, palladium rhodium, or titanium.

Any suitable number of electrodes 434 can be disposed on the lead including, for example, four, five, six, seven, eight, nine, ten, eleven, twelve, fourteen, sixteen, twenty-four, thirty-two, or more electrodes 434. In the case of paddle leads, the electrodes 434 can be disposed on the paddle body 444 in any suitable arrangement. In FIG. 4, the electrodes 434 are arranged into two columns, where each column has eight electrodes 434.

The electrodes of the paddle body 444 (or one or more lead bodies 446) are typically disposed in, or separated by, a non-conductive, biocompatible material such as, for example, silicone, polyurethane, polyetheretherketone (“PEEK”), epoxy, and the like or combinations thereof. The one or more lead bodies 446 and, if applicable, the paddle body 444 may be formed in the desired shape by any process including, for example, molding (including injection molding), casting, and the like. The non-conductive material typically extends from the distal ends of the one or more lead bodies 446 to the proximal end of each of the one or more lead bodies 446.

In the case of paddle leads, the non-conductive material typically extends from the paddle body 444 to the proximal end of each of the one or more lead bodies 446. Additionally, the non-conductive, biocompatible material of the paddle body 444 and the one or more lead bodies 446 may be the same or different. Moreover, the paddle body 444 and the one or more lead bodies 446 may be a unitary structure or can be formed as two separate structures that are permanently or detachably coupled together.

One or more terminals (e.g., 660 in FIGS. 6A-6B) are typically disposed along the proximal end of the one or more lead bodies 446 of the electrical stimulation system 400 (as well as any splitters, lead extensions, adaptors, or the like) for electrical connection to corresponding, connector contacts (e.g., 664 in FIGS. 6A-6B). The connector contacts are disposed in connectors (e.g., 445 in FIGS. 4-6B; and 672 FIG. 6B) which, in turn, are disposed on, for example, the implantable control module 402 (or a lead extension, a splitter, an adaptor, or the like). One or more electrically conductive wires, cables, or the like (i.e., “conductors”—not shown) extend from the terminal(s) to the electrode(s). In at least some embodiments, there is at least one (or exactly one) terminal conductor for each terminal which extends to at least one (or exactly one) of the electrodes.

The one or more conductors are embedded in the non-conductive material of the lead body 446 or can be disposed in one or more lumens (not shown) extending along the lead body 446. For example, any of the conductors may extend distally along the lead body 446 from the terminals 660.

FIG. 6A is a schematic side view of one embodiment of a proximal end of one or more elongated devices 600 configured and arranged for coupling to one embodiment of the connector 445. The one or more elongated devices may include, for example, one or more of the lead bodies 446 of FIG. 4, one or more intermediate devices (e.g., a splitter, the lead extension 624 of FIG. 6B, an adaptor, or the like or combinations thereof), or a combination thereof.

The connector 445 defines at least one port into which a proximal ends 446a, 446b of the elongated device 600 can be inserted, as shown by directional arrows 662a, 662b. In FIG. 6A (and in other figures), the connector housing 448 is shown having two ports 654a, 654b. The connector housing 448 can define any suitable number of ports including, for example, one, two, three, four, five, six, seven, eight, or more ports.

The connector 445 also includes one or more connector contacts, such as connector contact 664, disposed within each port 654a, 654b. When the elongated device 600 is inserted into the ports 654a, 654b, the connector contact(s) 664 can be aligned with the terminal(s) 660 disposed along the proximal end(s) of the elongated device(s) 600 to electrically couple the implantable control module 402 to the electrodes (134 of FIG. 4) disposed on the paddle body 445 of the lead 403. Examples of connectors in implantable control modules are found in, for example, U.S. Pat. Nos. 7,244,150 and 8,224,450, which are incorporated by reference.

FIG. 6B is a schematic side view of another embodiment of the electrical stimulation system 400. The electrical stimulation system 400 includes a lead extension 624 that is configured and arranged to couple one or more elongated devices 600 (e.g., one of the lead bodies 446 of FIGS. 4 and 5, the splitter 457 of FIG. 5, an adaptor, another lead extension, or the like or combinations thereof) to the implantable control module 402. In FIG. 6B, the lead extension 624 is shown coupled to a single port 654 defined in the connector 445. Additionally, the lead extension 624 is shown configured and arranged to couple to a single elongated device 600. In alternate embodiments, the lead extension 624 is configured and arranged to couple to multiple ports 654 defined in the connector 445, or to receive multiple elongated devices 600, or both.

A lead extension connector 672 is disposed on the lead extension 624. In FIG. 6B, the lead extension connector 672 is shown disposed at a distal end 676 of the lead extension 624. The lead extension connector 672 includes a connector housing 678. The connector housing 678 defines at least one port 664 into which terminal(s) 660 of the elongated device 600 can be inserted, as shown by directional arrow 638. The connector housing 678 also includes a plurality of connector contacts, such as connector contact 680. When the elongated device 600 is inserted into the port 630, the connector contacts 680 disposed in the connector housing 678 can be aligned with the terminal(s) 660 of the elongated device 600 to electrically couple the lead extension 624 to the electrodes (434 of FIGS. 4 and 5) disposed along the lead (403 in FIGS. 4 and 5).

In at least some embodiments, the proximal end of the lead extension 624 is similarly configured and arranged as a proximal end of the lead 403 (or other elongated device 600). The lead extension 624 may include one or more electrically conductive wires (not shown) that electrically couple the connector contact(s) 680 to a proximal end 648 of the lead extension 624 that is opposite to the distal end 676. The conductive wire(s) disposed in the lead extension 624 can be electrically coupled to one or more terminals (not shown) disposed along the proximal end 648 of the lead extension 624. The proximal end 648 of the lead extension 624 is configured and arranged for insertion into a connector disposed in another lead extension (or another intermediate device). As shown in FIG. 6B, the proximal end 648 of the lead extension 624 is configured and arranged for insertion into the connector 445.

The embodiments of FIGS. 4-6B illustrate a control module 402 with a connector 445 into which a proximal end portion of the lead or lead extension can be removably inserted. It will be recognized, however, that other embodiments of a control module and lead can have the lead or lead extension permanently attached to the control module. Such an arrangement can reduce the size of the control module as the conductors in the lead can be permanently attached to the electronic subassembly. It will also be recognized that, in at least some embodiments, more than one lead can be attached to a control module.

The above specification and examples provide a description of the manufacture and use of the invention. Since many embodiments of the invention can be made without departing from the spirit and scope of the invention, the invention also resides in the claims hereinafter appended.

What is claimed as new and desired to be protected by Letters Patent of the United States is:

1. An electrical stimulation system, comprising:
an implantable control module configured and arranged for implantation in a body of a patient and comprising an antenna, a secondary power source, and a processor coupled to the antenna and the secondary power source, wherein the control module is configured and arranged to provide electrical stimulation current to an electrical stimulation lead for stimulation of patient tissue; and
a primary power source configured and arranged to be worn or carried by the patient external to the body of the patient and to deliver power to the control module through the antenna, wherein the control module is configured and arranged to preferentially utilize power directly from the primary power source for the electrical stimulation current when the primary power source is available.

2. The electrical stimulation system of claim 1, further comprising an electrical stimulation lead coupleable, or coupled, to the control module and comprising at least one lead body having a distal end portion and a proximal end portion, a plurality of electrodes disposed along the distal end portion of the at least one lead body, a plurality of terminals disposed along the proximal end portion of the at least one lead body, and a plurality of conductors, the plurality of conductors electrically coupling the plurality of terminals to the plurality of electrodes.

3. The electrical stimulation system of claim 2, further comprising a lead extension coupleable between the electrical stimulation lead and the control module.

4. The electrical stimulation system of claim 1, wherein the secondary power source is configured and arranged to provide no more than four hours of continuous electrical stimulation current when fully charged.

5. The electrical stimulation system of claim 1, wherein the control module defines a port for receiving a proximal end of the electrical stimulation lead.

6. The electrical stimulation system of claim 1, wherein the primary power source comprises a processor configured and arranged to provide electrical stimulation current based on a set of stimulation parameters.

7. The electrical stimulation system of claim 1, further comprising an external programming unit configured and arranged for programming or modifying a set of stimulation parameters in the processor of the control module.

8. The electrical stimulation system of claim 1, wherein the control module is configured and arranged to solely utilize power directly from the primary power source for the electrical stimulation current when the primary power source is available.

9. The electrical stimulation system of claim 1, further comprising an electrical stimulation lead comprising at least one lead body having a distal end portion, a plurality of electrodes disposed along the distal end portion of the at least one lead body, and a plurality of conductors, the plurality of conductors electrically coupling the plurality of electrodes to the electronic subassembly of the control module.

10. An electrical stimulation system, comprising:
an implantable control module configured and arranged for implantation in a body of a patient and comprising an antenna, a secondary power source, and a processor coupled to the antenna and the secondary power source, wherein the control module is configured and arranged to provide electrical stimulation current to an electrical stimulation lead for stimulation of patient tissue; and
a primary power source configured and arranged to be worn or carried by the patient external to the body of the patient and to deliver power to the control module through the antenna, wherein the control module is configured and arranged to utilize power directly from the primary power source for the electrical stimulation current and the secondary power source has a storage capacity that is no more than 25% of a storage capacity of the primary power source.

11. The electrical stimulation system of claim 10, further comprising an electrical stimulation lead coupleable, or coupled, to the control module and comprising at least one lead body having a distal end portion and a proximal end portion, a plurality of electrodes disposed along the distal end portion of the at least one lead body, a plurality of terminals disposed along the proximal end portion of the at least one lead body, and a plurality of conductors, the plurality of conductors electrically coupling the plurality of terminals to the plurality of electrodes.

12. The electrical stimulation system of claim 11, further comprising a lead extension coupleable between the electrical stimulation lead and the control module.

13. The electrical stimulation system of claim 10, wherein the secondary power source is configured and arranged to provide no more than four hours of continuous electrical stimulation current when fully charged.

14. The electrical stimulation system of claim 10, further comprising an external programming unit configured and arranged for programming or modifying a set of stimulation parameters in the processor of the control module.

15. The electrical stimulation system of claim 10, wherein the control module is configured and arranged to solely utilize power directly from the primary power source for the electrical stimulation current when the primary power source is available.

16. An electrical stimulation system, comprising:
an implantable control module configured and arranged for implantation in a body of a patient and comprising an antenna, a secondary power source, and a processor coupled to the antenna and the secondary power source, wherein the control module is configured and arranged to provide electrical stimulation current to an electrical stimulation lead for stimulation of patient tissue;
a primary power source configured and arranged to be worn or carried by the patient external to the body of the patient and to deliver power to the control module through the antenna, wherein the control module is configured and arranged to utilize power directly from the primary power source for the electrical stimulation current;
an external antenna extending from the primary power source and comprising a distal end portion;
an external fixation element coupled to the distal end portion of the external antenna; and
an internal fixation element coupled to the implantable control module or the antenna of the control module, wherein the external and internal fixation elements are configured and arranged to hold the distal end portion of the external antenna to a skin of a patient at a preselected placement position when the internal fixation element is implanted in the patient near the preselected placement position.

17. The electrical stimulation system of claim 16, wherein the internal and external fixation elements each comprise a magnet.

18. The electrical stimulation system of claim 16, wherein the control module further comprises a housing with the antenna extending away from the housing, the antenna have a distal end portion, wherein the internal fixation element is disposed on a distal end portion of the antenna.

19. The electrical stimulation system of claim 16, further comprising an electrical stimulation lead coupleable, or coupled, to the control module and comprising at least one lead body having a distal end portion and a proximal end portion, a plurality of electrodes disposed along the distal end portion of the at least one lead body, a plurality of terminals disposed along the proximal end portion of the at least one lead body, and a plurality of conductors, the plurality of conductors electrically coupling the plurality of terminals to the plurality of electrodes.

20. The electrical stimulation system of claim 19, further comprising a lead extension coupleable between the electrical stimulation lead and the control module.
